(19) United States
(12) Patent Application Publication
(10) Pub. No.: US 2025/0195396 A1
(43) Pub. Date: Jun. 19, 2025

(54) TWO-STEP CLEANSING METHOD

(71) Applicant: L'OREAL, Paris (FR)

(72) Inventors: Xuefei LIU, Edison, NJ (US); Ritesh SINHA, Linden, NJ (US); Catherine CHIOU, Saddle Brook, NJ (US); Hy Si BUI, Piscataway, NJ (US)

(73) Assignee: L'OREAL, Paris (FR)

(21) Appl. No.: 18/544,330

(22) Filed: Dec. 18, 2023

(51) Int. Cl. A61K 8/92; A61K 8/31; A61K 8/81; A61K 8/86; A61Q 1/14

(52) U.S. Cl. CPC A61K 8/92; A61K 8/31; A61K 8/8152; A61K 8/86; A61Q 1/14; A61K 2800/262; A61K 2800/594; A61K 2800/884

(57) ABSTRACT

Described is a two-step method for removing makeup from skin. The method includes applying a first anhydrous composition to skin upon which makeup is present and subsequently applying a second anhydrous composition to the skin upon which the makeup and the first anhydrous composition are present. The first anhydrous composition include: (a) a hydrophobic polymer formed as a reaction product of a natural or food-derived oil and a methacrylate or acrylate polymer; (b) one or more solvents capable of solubilizing the hydrophobic polymer of (a); and (c) optionally, one or more alkanes. The second anhydrous composition includes one or more nonpolar oils in which the hydrophobic polymer of the first anhydrous composition is not soluble.

### FIELD OF THE DISCLOSURE

The instant disclosure is drawn to a method for cleansing skin using a two-step cleansing procedure. A first anhydrous composition is comprising a unique hydrophobic polymer is initially applied to skin, upon which makeup is present, followed by application of a second anhydrous composition.

### BACKGROUND

Many cosmetic compositions, including pigmented cosmetics such as foundations, mascaras, lipsticks, and eye shadows, have been formulated to have long-wear and transfer-resistance properties. Such properties are typically obtained by incorporating one or more film-forming agents into the compositions, such as oil-soluble film formers (or waxes), water-soluble film-formers, or resins (e.g., silicone resins like MQ resins). After application, the film-forming agents form a film that provides long-wear and transfer-resistance properties. However, these compositions and films can be difficult to remove, particularly if different types of film forming agents are included in the compositions.

Stubborn makeup may cause consumers to scrub, rub, and tug at the skin, which can disrupt skin's protective barrier, especially around delicate areas around the eyes. In addition, sleeping without removing makeup from the skin can cause a build-up and clog pores, leading to acne, inflammation, and skin irritation. Therefore, makeup removing products and procedures are needed.

Makeup removing products are formulated to break down and remove makeup, excess oil, and dirt from the skin. Several types of skin cleansing and makeup removing products have been developed, for example, rinsable anhydrous oils and gels, foaming creams, and lotions. Rinsable, anhydrous oils and gels have impart cleansing or makeup removing actions by virtue of the oils included in these formulations. The oils dissolve fatty residues and disperse make-up pigments. Although they are effective for dissolving certain elements of makeup compositions, they tend to be messy and difficult to use. Also, anhydrous makeup removing compositions tend to leave behind a greasy or oily residue, which can require additional effort to remove. Therefore, a need for improved makeup removing compositions and methods that are simple, effective, and gentle to the skin are needed.

### SUMMARY OF THE DISCLOSURE

The instant disclosure is drawn to a method for removing makeup from skin, including long-wear makeup. The method relies on a unique two-step process, wherein a first anhydrous composition is initially applied to the skin to dissolve and dislodge makeup present on the skin. A second anhydrous composition is subsequently used. The second anhydrous composition is applied without removing the first anhydrous composition from the skin. The second anhydrous composition is applied to the first anhydrous composition with which the makeup is dissolved or dislodged. Application of the second anhydrous composition results in the first anhydrous composition and the makeup dissolved within the first anhydrous composition, and the makeup dislodged by the first anhydrous composition, to solidify or coagulate for easy removal, for example, by peeling the material from the skin or by wiping or rinsing away the solidified or coagulated material. The process is surprisingly effective and provides the consumer with a unique cleansing experience.

The first anhydrous composition has a strong affinity for fatty materials, pigments, and other components of makeup, and efficiently solubilizes and dislodges the makeup, especially long-wear makeup. The first anhydrous composition and the solubilized or dislodged makeup is not appreciably soluble in the second anhydrous composition.

Therefore, application of the second anhydrous composition causes precipitation, aggregation, and solidification of the first anhydrous composition and makeup that has been absorbed or dislodged from the skin.

The first anhydrous composition typically includes:
(a) a hydrophobic polymer formed as a reaction product of a natural or food-derived oil and a methacrylate or acrylate polymer,
(b) one or more solvents capable of solubilizing the hydrophobic polymer of (a); and
(c) optionally, one or more alkanes.

The second anhydrous composition includes one or more nonpolar oils in which the hydrophobic polymer of (a) is not soluble. The second anhydrous composition can optionally include one or more additional components, for example, one or more oil thickening agents, solid fatty compounds, for example, solid waxes, and miscellaneous ingredients. The additional components, however, are often solubilized in the second anhydrous composition.

The hydrophobic polymer is a reaction product of a natural or food-derived oil and an acrylate or methacrylate polymer. According to certain embodiments, the hydrophobic polymer is a reaction product of a natural or food-derived oil and a methacrylate polymer. The natural or food-derived oil may be a drying oil or a semi-drying oil. Nonlimiting examples include linseed oil, sunflower oil, tung oil, fish oil, cottonseed oil, soybean oil, or combinations thereof. The methacrylate polymer may be formed from methacrylate monomers, for example, monomers selected from isobutyl methacrylate, methyl methacrylate, ethyl methacrylate, n-butyl methacrylate, and combinations thereof. In a preferred embodiment, the hydrophobic polymer is formed from a natural or food-derived oil and an isobutyl methacrylate polymer.

In various embodiments, the hydrophobic polymer is the reaction product of about 50 to about 85 parts by weight of the natural or food-derived oil and about 15 to about 50 parts by weight of the methacrylate or acrylate polymer. More specifically, the hydrophobic polymer may be the reaction product of about 72 to about 77 parts by weight of the natural or food-derived oil and about 23 to about 28 parts by weight of a methacrylate polymer. For example, the hydrophobic polymer may be the reaction product of linseed oil and poly(isobutyl methacrylate) in a suitable solvent, such as, for example, 2,2,4-trimethyl-1, 3-pentanediol monoisobutyrate. Preferably, the reaction product is formed from about 72 to about 77% of linseed oil and about 23 to about 28 parts by weight of isobutyl methacrylate polymer in a suitable solvent, such as 2,2,4-trimethyl-1, 3-pentanediol monoisobutyrate.

The hydrophobic polymer of (a) is solubilized in one or more solvents capable of solubilizing the hydrophobic polymer. A single solvent may be used or a combination of solvents, wherein the combination of solvents can solubilize the hydrophobic polymer. In various embodiments, the one or more solvents capable of solubilizing the hydrophobic polymer have a dispersion component (D), a polar component (β), a hydrogen bonding component (H), and a distance (Ra) per Hansen Solubility Parameters pf less than or equal to 13.4 MPa^{0.5}, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

Nonlimiting examples of solvents capable of solubilizing the hydrophobic polymer of (a) include polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof. Polycitronellol acetate, caprylic/capric triglyceride, isododecane, and mixtures thereof, are particularly useful.

The first anhydrous composition may optionally include one or more alkanes. Nonlimiting examples of alkanes include C₆-C₁₆ alkanes, for example, one or more alkanes selected from isododecane, isodecane, isohexadecane, n-heptane, n-octane, n-nonane, n-decane, n-undecane, n-dodecane, n-tetradecane, n-tridecane, n-tetradecane, n-pentadecane, or mixtures thereof.

The second anhydrous composition includes one or more nonpolar oils in which the hydrophobic polymer of (a) is not soluble. Nonlimiting examples include squalane, isoparaffin (C12-C14), cycloparaffin, polydecene, polydimethylsiloxane, squalene, hydrogenated polyisobutene, isohexadecane, mineral oil, isoeicosane, ethoxydiglycol oleate, decyl olivate, or mixtures thereof.

The second anhydrous composition optionally includes one or more oil thickening agents that thicken the second anhydrous composition. Nonlimiting examples of oil thickening agents include polymers, gums, organoclays, polyethylenes, silica, or mixtures thereof. More specific but nonlimiting examples include acrylate copolymers, hectorite gel, silica, silica dimethyl silylate, behenate, glyceryl dibehenate, behenyl behenate, or mixtures thereof.

The second anhydrous composition optionally includes one or more solid fatty compounds. Nonlimiting examples of solid fatty compounds include solid fatty acids, solid fatty alcohol, solid waxes, and the like. The solid fatty compounds are solid at 25° C. and atmospheric pressure. Nonetheless, the solid fatty compounds are typically solubilized in the second anhydrous composition, i.e., they do not exist as solids when formulated in the second anhydrous composition. Nonlimiting examples of solid fatty compounds include candelilla wax, carnauba wax, castor wax, beeswax, lanolin Wax, ozokerite wax, microcrystalline wax, sunflower wax, tribehenin, cetyl alcohol, stearyl alcohol, cetearyl alcohol, lanolin alcohol, or mixtures thereof.

The first anhydrous composition, the second anhydrous composition, or both the first and second anhydrous composition may be transparent.

The first anhydrous composition and the second anhydrous composition may be provided as a kit. Such kits include:
(i) first anhydrous comprising:
   (a) a hydrophobic polymer formed as a reaction product of a natural or food-derived oil and a methacrylate or acrylate polymer;
   (b) one or more solvents capable of solubilizing the hydrophobic polymer of (i)(a); and
   (c) optionally, one or more alkanes; and
(ii) a second anhydrous composition comprising:
   (a) one or more nonpolar oils in which the hydrophobic polymer of (i)(a) is not soluble;
   (b) optionally, one or more thickening agent capable of thickening the second anhydrous composition; and
   (c) optionally, one or more solid fatty compounds that are soluble in the second anhydrous composition;
      wherein the first anhydrous composition and the second anhydrous composition are separately contained.

The methods of the disclosure include methods for cleansing the skin and methods for removing makeup from skin, in particular, long-wear makeup. Nonlimiting examples of makeup that can be removed include mascara, lipstick, rouge, eyeliner, eye shadow, foundation, blush, and the like.

### DETAILED DESCRIPTION OF THE DISCLOSURE

The present disclosure is drawn to methods and compositions for cleansing the skin and removing makeup from the skin, including long-wear makeup. A first anhydrous composition and a second anhydrous composition are used together in a method that effectively and efficiently cleanses the skin and removes makeup from the skin. The first anhydrous composition is initially applied to the skin to dissolve and dislodge makeup present on the skin. A second anhydrous composition is subsequently applied, without first removing the first anhydrous composition from the skin. Application of the second anhydrous composition results in the first anhydrous composition, and the makeup, to solidify and coagulate for easy removal from the skin, for example, by peeling the material from the skin or by wiping or rinsing away the solidified or coagulated residue. The process is surprisingly effective and provides the consumer with a unique cleansing experience.

The method for removing makeup from skin comprises:
(i) applying a first anhydrous composition to skin upon which makeup is present, the first anhydrous composition comprising:
   (a) a hydrophobic polymer formed as a reaction product of a natural or food-derived oil and a methacrylate or acrylate polymer;
   (b) one or more solvents capable of solubilizing the hydrophobic polymer of (i)(a); and
   (c) optionally, one or more alkanes;
(ii) applying a second anhydrous composition to skin upon which the makeup and the first anhydrous composition are present, the second anhydrous composition comprising:
   (a) one or more nonpolar oils in which the hydrophobic polymer of (i)(a) is not soluble;
   (b) optionally, one or more thickening agents capable of thickening the second anhydrous composition;
   (c) optionally, one or more solid fatty compounds, wherein the one or more solid fatty compounds is soluble in the second anhydrous composition.

The first anhydrous composition, the second anhydrous composition, or both the first and second anhydrous composition can be transparent or translucent.

The first anhydrous composition is applied to skin upon which makeup is present. It can be rubbed over the skin and makeup, for example, with an individual's hand or with a cloth or other substrate. A rubbing action helps dissolve and dislodge makeup adhered to the skin. The first anhydrous composition is typically applied to the skin, upon which makeup is present, and optionally rubbed or mixed with makeup on the skin. The first anhydrous composition can remain on the skin with the makeup for a period of time, for example, for about 10 seconds to about 10 minutes. The first anhydrous composition is effective for dissolving and dislodging the makeup and therefore a long period of time is not needed.

The first anhydrous composition immediately begins dissolving and dislodging makeup adhered to the skin upon application and the process is expedited by rubbing or mixing the first anhydrous composition with the makeup on the skin. The second anhydrous composition is typically applied immediately or shortly after application of the first anhydrous composition. For example, the second anhydrous composition may be applied within about 10 minutes, with 5 minutes, or within 1 minute after application of the first anhydrous composition.

### (i) First Anhydrous Composition

A primary role of the first anhydrous composition is to solubilize, dislodge, and to adhere to makeup and ingredients of the makeup present on the skin. This is achieved, at least in part, with a hydrophobic polymer that is as a reaction product of a natural or food-derived oil and a methacrylate or acrylate polymer. The first anhydrous composition is typically a solution comprising the hydrophobic polymer. The one or more solvents capable of solubilizing the hydrophobic polymer also assist in dissolving and removing makeup from the skin.

(i)(a) Hydrophobic Polymer

The hydrophobic polymer is a reaction product of a natural or food-derived oil (oil component) and an acrylate component. In particular, the natural or food-derived oil may be a drying oil, preferably linseed oil. The reaction product may include an isobutyl methacrylate backbone with a plurality of linseed oil side chains. Preferably, the reaction product is a product sold under the MYCELX® brand from MYCELX Technologies Corporation of Gainesville, Georgia. See U.S. Pat. No. 5,698,139 for a description of MYCELX materials, which is incorporated herein by reference in its entirety.

The hydrophobic polymer is comprised of an oil component and a polymer component, typically reacted in a solvent. In a preferred embodiment, the hydrophobic polymer is a reaction product of linseed oil and poly(isobutyl methacrylate), in a solvent such as 2,2,4-trimethyl-1,3-pentanediol-monoisobutyrate as a solvent.

The oil component is derived from glycerin and carboxylic acids, such as linseed fatty acid to form monoglycerides, diglycerides, and triglycerides. The oil component is preferably derived from plant/vegetable or natural origin. Vegetable oils are obtained by cold pressing the seeds of a plant to obtain the oil contained therein. Of the vegetable oils, drying oils such as linseed and tung oil, semi-drying oils such as soybean and cotton seed oil, and non-drying oils such as coconut oil may be used as the oil component. The oil component typically forms about 72% to 77%, or most preferably, 74.62%, of the hydrophobic polymer (e.g., linseed oil/isobutyl methacrylate).

The polymer component may be derived from a and β-unsaturated carbonyl compounds. The polymer component is the resultant product of a monomer which is an ester of an acrylic acid, crotonic acid, isocrotonic acid, methacrylic acid, sorbic acid, cinnamic acid, maleic acid, fumaric acid, and methyl methacrylic acid. Nonlimiting examples of useful polymers which cover any number of reaction possibilities between the esters of such compounds include acrylate polymers, methyl methacrylate polymers, methyl/n-butyl methacrylate polymers, methacrylate copolymers, ethyl methacrylate, n-butyl methacrylate, isobutyl methacrylate, n-butyl/isobutyl methacrylate copolymers, or combinations thereof. Preferably the polymer is poly(isobutyl methacrylate).

The hydrophobic polymer is a reaction product typically formed in a liquid solvent able to dissolve or dilute the polymer component (poly(oil/polymer)) and the hydrophobic polymer. The solvent, or diluent should generally comprise any liquid or mixture of liquids that is able to dissolve or dilute hydrophobic polymer product. The solvent/diluent can control the evaporation, desired flow, and coalescing of the hydrophobic polymer. The solvent may be, for example, an aliphatic hydrocarbon, aromatic hydrocarbon, alcohols, ketones, ethers, aldehydes, phenols, carboxylic acids, carboxylates, synthetic chemicals and naturally occurring substances. Preferably the solvent is 2,2,4-trimethyl-1,3-pentanediol-monoisobutyrate. Hydrophobic polymers according to the instant disclosure and methods for making them are described, for example, in U.S. Pat. Nos. 5,437,793, 5,698,139, 5,837,146, 5,961,823, 6,180,010, 6,475,393, and 6,805,727, which are incorporated herein by reference in their entireties. The preferred hydrophobic polymer may be designated as poly(linseed oil/isobutyl methacrylate).

The amount of hydrophobic polymer in the first anhydrous composition will vary. Nonetheless, the total amount of the hydrophobic polymer in the first anhydrous composition is typically from about 1 to about 40 wt. %, based on the total weight of the first anhydrous composition. In further embodiment, the total amount of the hydrophobic polymer is from about 1 to about 35 wt. %, about 1 to about 30 wt. %, about 5 to about 40 wt. %, about 5 to about 35 wt. %, about 5 to about 30 wt. %, about 5 to about 26 wt. %, about 10 to about 40 wt. %, about 10 to about 35 wt. %, about 10 to about 30 wt. %, about 10 to about 26 wt. %, about 15 to about 40 wt. %, about 15 to about 35 wt. %, about 15 to about 30 wt. %, about 15 to about 26 wt. %, about 20 to about 40 wt. %, about 20 to about 35 wt. %, about 20 to about 30 wt. %, about 20 to about 26 wt. %, about 25 to about 40 wt. %, about 25 to about 35 wt. %, about 25 to about 30 wt. %, or about 25 to about 28 wt. %, based on the total weight of the first anhydrous composition.

(i)(b) Solvent Capable of Solubilizing (i)(a)

The hydrophobic polymer of (i)(a) is typically dissolved in one or more solvents capable of solubilizing the hydrophobic polymer. The solvent may include one or more solvents used to generate the hydrophobic polymer of (i)(a), i.e., used in generating the reaction product (hydrophobic polymer), such as, 2,2,4-trimethyl-1,3-pentanediol-monoisobutyrate. The solvent may be a single solvent or a plurality of solvents. For example, in various embodiments, solvents capable of solubilizing the hydrophobic polymer have a dispersion component (D), a polar component (β), a hydrogen bonding component (H), and a distance (Ra) of less than or equal to 13.4 MPa^{0.5} per the Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

In a preferred embodiment, the one or more solvents have a dispersion component (D), a polar component (β), a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}.

The solvent may be an oil. The term “oil” is intended to mean a non-aqueous compound that is non-miscible in water and liquid at 25° C. and atmospheric pressure (760 mmHg; 1.013×10⁵ Pa). The solvent may be a non-silicone oil (e.g., an oil that does not contain silicon atoms, and in particular does not contain Si—O groups). Nonlimiting examples of useful solvents include caprylic/capric triglyceride, isopropyl myristate, polycitronellol acetate, and mixtures thereof. The solvents may include acetone. The solvents may include oleic acid. The solvents may include an oleic acid containing oil (such as a vegetable oil). Table 1 below, shows values of D, P, and H, as well as Ra values for the allowable range as well as the preferred range, for several preferred solvents.

TABLE 1

| | | | | Ra | Ra |
|---|---|---|---|---|---|
| | | | | (Allowable | (Preferred |
| Solvent (b) | D | P | H | Range) | Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl Myristate | 15.9 | 2.1 | 2.8 | 10.70 | 9.41 |
| Isopropyl Alcohol | 15.8 | 6.1 | 6.4 | 7.02 | 5.54 |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol Acetate | 16.4 | 3 | 4.2 | 9.02 | 7.76 |
| Acetone | 15.5 | 10.4 | 7 | 8.61 | 7.38 |
| Oleic Acid | 16 | 2.8 | 6.2 | 7.27 | 5.98 |

In some embodiments, if oleic acid is utilized, at least some of the oleic acid may be provided by a vegetable oil. The vegetable oil may be a seed or nut oil. The vegetable oil may have an oleic acid content of at least 20% by weight of the vegetable oil. The vegetable oil may include sunflower oil, soybean oil, macadamia nut oil, and/or avocado oil. In some embodiments, the composition may include macadamia nut oil, and may be free, or substantially free, of other vegetable oils.

For purposes of the instant disclosure, the one or more of the solvents capable of solubilizing the hydrophobic polymer of (i)(a) may not individually solubilize the hydrophobic polymer. However, when combined with other solvents, the combination solubilizes the hydrophobic polymer. Thus, when referring to a total amount of one or more solvents capable of solubilizing the hydrophobic polymer, the inclusion of all solvents that in combination solubilize the hydrophobic polymer is intended, even if one or more solvents in the combination do not individually solubilize the hydrophobic polymer.

Nonlimiting examples of solvents useful for solubilizing the hydrophobic polymer of (i)(a), individually, or in combination with other solvents, include polycitronellol acetate, caprylic/capric triglyceride, isododecane, isohexadecane, tetradecane, isopropyl myristate, octyldodecanol, ethanol, phenoxyethanol, castor oil, and mixtures thereof. In a preferred embodiment, at least one of the one or more solvents capable of solubilizing the hydrophobic polymer are selected from caprylic/capric triglyceride, polycitronellol acetate, isododecane, and mixtures thereof. In another preferred embodiment, at least one of the one or more solvents capable of solubilizing the hydrophobic polymer is polycitronellol acetate, caprylic/capric triglyceride, isododecane, or mixtures thereof.

Further nonlimiting solvents that individually or in combination with other solvents are useful for solubilizing the hydrophobic polymer of (a) include dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglyceride, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof.

In various embodiments, the one or more oils capable of solubilizing the hydrophobic polymer of (i)(a) are selected from polar oils and medium polar oils. The polarity of an oil is defined as the polarity index (interfacial tension) of the oil with respect to water. The polarity may be determined using a ring tensiometer (e.g., Krüss K 10), which measures the interfacial tension in mN/m in analogy to the ASTM method D971-99a (2004).

Interfacial tension is the force which acts on an imaginary line one meter in length in the interface between two phases. The physical unit for this interfacial tension is conventionally calculated from the force/length relationship and is usually expressed in mN/m (millinewtons divided by meters). For the purposes of the present disclosure, medium polar oils (or mid-polar oils) have an interfacial tension (Polarity Index) of 23 mN/m to 37 mN/m, and polar oils have an interfacial tension (Polarity Index) less than 23 mN/m. Nonlimiting examples of medium polar oils (or mid-polar oils) and polar oils are set forth below in Table A.

TABLE A

| | |
|---|---|
| | Polarity Index |
| Oil (CTFA Name) | [mN/m] |

Medium Polar Oils (23 mN/m-37 mN/m)

| | |
|---|---|
| Isocetyl Palmitate (Tegosoft ® HP) | 36.2 |
| Isocetyl Palmitate (Isofol Ester ® 1693) | 33.5 |
| Cyclopentasiloxane | 32.3 |
| Dicaprylyl carbonate | 31.7 |
| Octyl Isostearate | 31.6 |
| Trimethylhexyl Isononanoate | 31.1 |
| 2-Ethylhexyl Isononanoate | 31.0 |
| Dicaprylyl ether | 30.9 |
| Dihexyl carbonate | 30.9 |
| Polydecene | 30.1 |
| Octyl Cocoate | 30.0 |
| Isodecyl Neopentanoate | 29.9 |
| Isohexy Decanoate (Dermol ® IHD) | 29.7 |
| Isodecyl Octanoate ( Dermol ® 108) | 29.6 |
| Dihexyl Ether | 29.2 |
| Isodecyl 3,5,5 Trimethyl Hexanoate (Dermol 109) | 29.1 |
| Oleyl Erucate (Cetiol J600 ®) | 27.1 |
| Passiflora Incarnata Oil (Cegesoft ® PFO) | 27.2 |
| Jojoba oil | 26.2 |
| Octyl palmitate | 23.1 |
| Macadamia nut oil | 22.1 |
| Isopropyl stearate | 21.9 |
| Rapeseed oil | 21.9 |
| Hexyl Decanol (Isofol ® 16) | 24.3 |
| Isotridecyl 3,5,5 Trimethylhexanonanoate | 24.5 |
| (Dermol ® 139) | |
| Mixed decanoyl and octanoyl glycerides | 25.3 |
| (Myritol ® 312) | |
| 2-Ethylhexanoic Acid 3,5,5 Trimethyl | 26.2 |
| Ester (Dermol ® 98) | |
| Cetystearyl octanoate | 28.6 |
| Dimethicone (silicon oil 20 ct) | 26.6 |
| Isopropyl palmitate | 25.2 |
| Octyldodecanol | 24.8 |
| Dioctyl adipate (ARLAMOL ® DOA) | 24.5 |
| Isopropyl myristate | 24.2 |
| Octyl palmitate (2-ethylhexyl palmitate) | 23.1 |

Polar Oils (<23 mN/m)

| | |
|---|---|
| Octyldodeceyl Myristate | 22.1 |
| Butyl Octanoic Acid (ISOCARB ® 12) | 22.1 |
| Isopropyl stearate | 21.9 |
| Caprylic/capric triglyceride | 21.3 |
| Isopropyl isostearate | 21.2 |
| Jojoba oil | 20.8 |
| Cyclomethicone (ARLAMOL ® D4) | 20.6 |
| Groundnut oil | 20.5 |
| Almond oil | 20.3 |
| Sunflower oil | 19.3 |
| Decyl oleate | 18.7 |
| Avocado oil | 18.3 |
| Olive oil | 16.9 |
| Dibutyl adipate | 14.3 |
| Castor oil | 13.7 |
| Calendula oil | 11.1 |
| Wheatgerm oil | 8.3 |
| Almond oil | 20.3 |
| Sunflower oil | 19.3 |
| Decyl oleate | 18.7 |
| Avocado oil | 18.3 |
| Olive oil | 16.9 |
| Castor oil | 13.7 |
| Calendula oil | 11.1 |
| Wheatgerm oil | 8.3 |
| Propylene glycol monoisostearate | 6.2 |
| Cocoglycerides | 5.1 |

The total amount of the one or more solvents capable of solubilizing the hydrophobic polymer of (i)(a) are often the predominant component of the first anhydrous composition. Therefore, the one or more solvents may constitute from about 35 to about 99 wt. % of the first anhydrous composition. In further embodiments, the first anhydrous composition includes from about 35 to about 95 wt. %, about 35 to about 90 wt. %, about 35 to about 85 wt. %, about 35 to about 80 wt. %, about 35 to about 75 wt. %, about 35 to about 70 wt. %, about 35 to about 65 wt. %, about 35 to about 60 wt. %, about 45 to about 99 wt. %, about 45 to about 95 wt. %, about 45 to about 90 wt. %, about 45 to about 85 wt. %, about 45 to about 80 wt. %, about 45 to about 75 wt. %, about 45 to about 70 wt. %, about 45 to about 65 wt. %, about 45 to about 60 wt. %, about 55 to about 99 wt. %, about 55 to about 95 wt. %, about 55 to about 90 wt. %, about 55 to about 85 wt. %, about 55 to about 80 wt. %, about 55 to about 75 wt. %, about 55 to about 70 wt. %, about 55 to about 65 wt. %, about 55 to about 60 wt. % of the one or more solvents capable of solubilizing the hydrophobic polymer of (i)(a), based on the total weight of the first anhydrous composition.

(i)(c) Alkanes

The first anhydrous composition may optionally include one or more alkanes. The one or more alkanes may be considered part of the one or more solvents capable of solubilizing the hydrophobic polymer of (i)(a) or may be accounted for separately considering the hydrophobic polymer of (i)(a) is not necessarily soluble in alkanes but can be solubilized when one or more alkanes are combined with other solvents capable of solubilizing the hydrophobic polymer of (i)(a).

Alkanes are a series of compounds that contain carbon and hydrogen atoms with single covalent bonds. Squalane is an example of an alkane. Alkanes are nonpolar because they contain nonpolar carbon-carbon and carbon-hydrogen bonds. They are therefore not soluble in water, and since they are generally less dense than water, they typically float on water. Due to their lack of polarity, they are individually not capable of solubilizing the hydrophobic polymers of (i)(a). Nonetheless, having an amount of an alkane in the first anhydrous composition, in combination with solvents that are individually capable of solubilizing the hydrophobic polymer of (i)(a), results in the first anhydrous composition being able to solubilize the hydrophobic polymer of (i)(a) and contributes to makeup removing properties, as isododecane readily dissolves long-wear makeup. However, it also helps the second anhydrous composition expeditiously solidify or coagulate the hydrophobic polymer of (i)(a) when the second anhydrous composition is applied. A smaller amount of the second anhydrous composition is needed to transform the hydrophobic polymer because the first anhydrous composition already includes an amount of alkane in which the hydrophobic polymer of (i)(a) is not individually soluble, and therefore includes lower amounts of individual solvents capable of solubilizing the hydrophobic polymer of (i)(a).

Nonlimiting examples of alkanes include linear and branches alkanes having from 6 to 16 carbon atoms. Nonlimiting examples include isododecane, isodecane, isohexadecane, n-heptane, n-octane, n-nonane, n-decane, n-undecane, n-dodecane, n-tetradecane, n-tridecane, n-tetradecane, n-pentadecane, or mixtures thereof.

The total amount of the one or more alkanes in the first anhydrous composition, if present, will vary. Nonetheless, in various embodiments, the first anhydrous composition includes from about 1 to about 30 wt. % of one or more alkanes. In further embodiments, the first anhydrous composition includes from about 1 to about 25 wt. %, about 1 to about 20 wt. %, about 1 to about 15 wt. %, about 1 to about 10 wt. %, about 2 to about 30 wt. %, about 2 to 25 wt. %, about 2 to about 20 wt. %, about 2 to about 15 wt. %, about 2 to about 10 wt. %, about 5 to about 30 wt. %, about 5 to about 25 wt. %, about 5 to about 20 wt. %, about 5 to about 15 wt. %, about 5 to about 10 wt. %, about 8 to about 30 wt. %, about 8 to about 25 wt. %, about 8 to about 20 wt. %, about 8 to about 15 wt. %, about 10 to about 30 wt. %, about 10 to about 25 wt. %, about 10 to about 20 wt. %, or about 10 to about 15 wt. %, based on the total weight of the first anhydrous composition.

### Oil Thickening Agents

The first anhydrous composition may include one or more oil thickening agents. An “oil thickening agent” is a material which when combined with the solvents of the first anhydrous composition results in a thickening of the first anhydrous composition. Nonlimiting examples of oil thickening agents include polymers, gums, organoclays, polyethylenes, silica, or combinations thereof. More specific but nonlimiting examples include acrylate copolymer, hectorite gel, guar gum, cellulose gum, Caesalpinia Spinosa gum, gum Arabic, starch, silica, silica dimethyl silylate, behenate, glyceryl dibehenate, behenyl behenate, or mixtures thereof. In various embodiments, polyalcohol acrylates may be useful, for examples, poly C10-30 alkyl acrylate. The oil thickening agents can be selected from semi-crystalline or crystalline polymers and/or semi-crystalline or crystalline waxes.

In one embodiment, one or more oil thickening agent are selected from polymeric thickening agents and inorganic thickening agents. In one embodiment, the at least one polymeric thickening agent can be an amorphous polymer formed by polymerization of an olefin. In a further embodiment, the olefin can be an ethylenically unsaturated elastomer monomer. Examples of olefins include, but are not limited to, ethylenic carbide monomers, for example those comprising one or two ethylenic unsaturations, comprising from 2 to 5 carbon atoms, such as ethylene, propylene, butadiene, or isoprene.

In at least one embodiment, the at least one polymeric thickening agent of oils is capable of thickening or gelling the organic phase of the composition. As used herein, “amorphous polymer” means a polymer which does not have a crystalline form.

In another embodiment, the oil thickening agents may be polycondensation products of the polyamide type resulting from condensation between (a) at least one acid chosen from dicarboxylic acids comprising at least 32 carbon atoms, such as the dimeric fatty acids and (β) alkylenediamines, such as ethylenediamine, wherein the polymeric polyamide comprises at least one terminal carboxylic acid group esterified or amidated with at least one monoalcoholor one monoamine comprising from 12 to 30 linear and saturated carbon atoms, for example ethylenediamine/stearyl dilinoleate copolymers such as that marketed under the name Uniclear 100 VG® by Arizona Chemical.

The oil thickening agent may be selected from inorganic thickening agents of oils, such as organophilic clay or at least one pyrogenic silica. As used herein, “organophilic clays” mean clays modified with chemical compounds rendering the clay capable of swelling in oily media. The clays are products already well known in themselves, which are for example described in the book “Mineralogy of clays, S. Caillère, S. Hénin, M. Rautureau, 2nd edition 1982, Masson”, the teaching of which is incorporated herein by reference in its entirety. In one embodiment, the at least one clay is a silicate comprising a cation which may be chosen from the cations of calcium, magnesium, aluminium, sodium, potassium, and lithium. Examples of such clays include, but are not limited to, the clays of the smectite family such as montmorillonites, hectorites, bentonites, beidellites, saponites, and the vermiculite family, stevensite, and chlorites. These clays can be of natural or synthetic origin.

The total amount of the one or more oil thickening agents, if present, will vary. Nonetheless, in certain embodiments, the first anhydrous composition includes from about 0.1 to about 10 wt. % of one or more oil thickening agents, based on the total weight of the second anhydrous composition. In further embodiment, the total amount of the one or more oil thickening agents is from about 0.1 to about 5 wt. %, about 0.1 to about 3 wt. %, about 0.5 to about 10 wt. %, about 0.5 to about 5 wt. %, about 0.5 to about 3 wt. %, about 1 to about 10 wt. %, about 1 to about 5 wt. %, or about 1 to about 3 wt. %, based on the total weight of the first anhydrous composition.

### Miscellaneous Ingredients

The first anhydrous composition may optionally include one or more miscellaneous ingredients. Miscellaneous ingredients are ingredients that are compatible with the first anhydrous composition and do not disrupt or materially affect the basic and novel properties of the first anhydrous composition. Nonlimiting examples of ingredients include preservatives, fragrances, pH adjusters, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the first anhydrous composition, if present, will vary. Nonetheless, the first anhydrous composition may include from about 0.1 to about 15 wt. % of the one or more miscellaneous ingredients, based on the total weight of the first anhydrous composition. In further embodiments, the first anhydrous composition includes from about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of one or more miscellaneous ingredients, based on the total weight of the first anhydrous composition.

### (ii) Second Anhydrous Composition

A primary role of the second anhydrous composition is to solidify, congeal, and precipitate the hydrophobic polymer (i)(a) of the first anhydrous composition and the makeup that interacts with the hydrophobic polymer. The hydrophobic polymer has an affinity for makeup or elements in the makeup and therefore sequesters or attaches to the makeup and the elements of the makeup. Upon application of the second anhydrous composition and the congealing, hardening, or precipitation of the hydrophobic polymer (and the makeup and elements of makeup with which it interacts), the solidified or agglomerated material can be easily removed from the skin, for example, with rinsing, wiping, or rubbing. A significant amount of the second anhydrous composition is one or more nonpolar oils in which the hydrophobic polymer of (i)(a) is not soluble.

(ii)(a) Nonpolar Oils

Nonlimiting examples of nonpolar oils include squalane, isoparaffin (C12-C14), cycloparaffin, polydecene, polydimethylsiloxane, squalene, hydrogenated polyisobutene, isohexadecane, mineral oil, isoeicosane, ethoxydiglycol oleate, decyl olivate, or mixtures thereof.

In various embodiments, the nonpolar oil may be a nonpolar oil having a polarity index (interfacial tension) of at greater than 37 mN/m. The polarity of an oil is defined as the polarity index (interfacial tension) of the oil with respect to water. The polarity may be determined using a ring tensiometer (e.g., Krüss K 10), which measures the interfacial tension in mN/m in analogy to the ASTM method D971-99a (2004).

Interfacial tension is the force which acts on an imaginary line one meter in length in the interface between two phases. The physical unit for this interfacial tension is conventionally calculated from the force/length relationship and is usually expressed in mN/m (millinewtons divided by meters). For the purposes of the present disclosure, nonpolar oils have an interfacial tension (Polarity Index) of greater than 37 mN/m. Nonlimiting examples of nonpolar oils are provided below in Table B.

TABLE B

| | |
|---|---|
| | Polarity Index |
| Oil (CTFA Name) | [mN/m] |

Nonpolar Oils (>40 mN/m)

| | |
|---|---|
| Isoparaffin (C12-C14) | 53.0 |
| Cycloparaffin | 49.1 |
| Polydecene | 46.7 |
| Polydimethylsiloxane | 46.5-40.9 ¹ |
| Squalane (Squalan ®) | 46.2 |
| Hydrogenated polyisobutene (Luvitol Lite ®) | 44.7 |
| Isohexadecane (ARLAMOL ® ND) | 43.8 |
| Mineral oil (Paraffin oil perliquidum) | 43.7 |
| Isoeicosane | 41.9 |
| Ethoxydiglycol Oleate | 40.5 |
| Decyl Olivate | 40.3 |

¹ Polydimethylsiloxane polymers have different polarity depending on polymer length.

The total amount of the one or more nonpolar oils in the second anhydrous composition will vary depending on amounts of other ingredients in the second anhydrous composition. Nonetheless, the total amount of the one or more nonpolar oils can be from about 50 wt. % to about 99.9 wt. %, based on the total weight of the second anhydrous composition. In further embodiments, the total amount of the one or more nonpolar oils is from about 50 to about 99 wt. %, about 50 to about 95 wt. %, about 50 to about 90 wt. %, about 50 to about 80 wt. %, about 50 to about 70 wt. %, about 50 to about 60 wt. %, about 60 to about 99.9 wt. %, about 60 to about 99 wt. %, about 60 to about 95 wt. %, about 60 to about 90 wt. %, about 60 to about 80 wt. %, about 60 to about 70 wt. %, about 70 to about 99.9 wt. %, about 70 to about 99 wt. %, about 70 to about 95 wt. %, about 70 to about 90 wt. %, about 70 to about 80 wt. %, about 80 to about 99.9 wt. %, about 80 to about 99 wt. %, about 80 to about 95 wt. %, or about 80 to about 90 wt. %, based on the total weight of the second anhydrous composition.

(ii)(b) Oil Thickening Agents

The term “oil thickening agent” means a material which when combined with the nonpolar oil of the second anhydrous composition results in a thickening of the second anhydrous composition. Nonlimiting examples of oil thickening agents include polymers, gums, organoclays, polyethylenes, silica, or combinations thereof. More specific but nonlimiting examples include acrylate copolymer, hectorite gel, guar gum, cellulose gum, Caesalpinia Spinosa gum, gum Arabic, starch, silica, silica dimethyl silylate, behenate, glyceryl dibehenate, behenyl behenate, or mixtures thereof. In various embodiments, polyalcohol acrylates may be useful, for examples, poly C10-30 alkyl acrylate. The oil thickening agents can be selected from semi-crystalline or crystalline polymers and/or semi-crystalline or crystalline waxes.

In one embodiment, one or more oil thickening agent are selected from polymeric thickening agents and inorganic thickening agents. In one embodiment, the at least one polymeric thickening agent can be an amorphous polymer formed by polymerization of an olefin. In a further embodiment, the olefin can be an ethylenically unsaturated elastomer monomer. Examples of olefins include, but are not limited to, ethylenic carbide monomers, for example those comprising one or two ethylenic unsaturations, comprising from 2 to 5 carbon atoms, such as ethylene, propylene, butadiene, or isoprene.

In at least one embodiment, the at least one polymeric thickening agent of oils is capable of thickening or gelling the organic phase of the composition. As used herein, “amorphous polymer” means a polymer which does not have a crystalline form.

In another embodiment, the oil thickening agents may be polycondensation products of the polyamide type resulting from condensation between (a) at least one acid chosen from dicarboxylic acids comprising at least 32 carbon atoms, such as the dimeric fatty acids and (β) alkylenediamines, such as ethylenediamine, wherein the polymeric polyamide comprises at least one terminal carboxylic acid group esterified or amidated with at least one monoalcoholor one monoamine comprising from 12 to 30 linear and saturated carbon atoms, for example ethylenediamine/stearyl dilinoleate copolymers such as that marketed under the name Uniclear 100 VG® by Arizona Chemical.

The oil thickening agent may be selected from inorganic thickening agents of oils, such as organophilic clay or at least one pyrogenic silica. As used herein, “organophilic clays” mean clays modified with chemical compounds rendering the clay capable of swelling in oily media. The clays are products already well known in themselves, which are for example described in the book “Mineralogy of clays, S. Caillère, S. Hénin, M. Rautureau, 2nd edition 1982, Masson”, the teaching of which is incorporated herein by reference in its entirety. In one embodiment, the at least one clay is a silicate comprising a cation which may be chosen from the cations of calcium, magnesium, aluminium, sodium, potassium, and lithium. Examples of such clays include, but are not limited to, the clays of the smectite family such as montmorillonites, hectorites, bentonites, beidellites, saponites, and the vermiculite family, stevensite, and chlorites. These clays can be of natural or synthetic origin.

The total amount of the one or more oil thickening agents, if present, will vary. Nonetheless, in certain embodiments, the second anhydrous composition includes from about 0.1 to about 10 wt. % of one or more oil thickening agents, based on the total weight of the second anhydrous composition. In further embodiment, the total amount of the one or more oil thickening agents is from about 0.1 to about 5 wt. %, about 0.1 to about 3 wt. %, about 0.5 to about 10 wt. %, about 0.5 to about 5 wt. %, about 0.5 to about 3 wt. %, about 1 to about 10 wt. %, about 1 to about 5 wt. %, or about 1 to about 3 wt. %, based on the total weight of the second anhydrous composition.

(ii)(c) Solid Fatty Compounds

The second anhydrous composition may optionally include one or more solid fatty compounds. A solid fatty compound has a melting point above 25° C. at atmospheric pressure, i.e., they are solid at 25° C. and atmospheric pressure. Although the second anhydrous composition may include one or more fatty compounds, the one or more fatty compounds is typically not solid when incorporated into the second anhydrous composition. The one or more solid fatty compounds is usually dissolved by the other components of the second anhydrous composition, for example, by the one or more nonpolar oils.

Nonlimiting examples of solid fatty compounds include solid animal and solid vegetable oils and fats, fully hydrogenated or partially hydrogenated vegetable and animal oils and fats, saturated fatty acids, partially hydrogenated or fully hydrogenated fatty acids, fatty acid esters, saturated, partially hydrogenated or fully hydrogenated monoglycerides, diglycerides and triglycerides, phospholipids, lecithins, partially hydrogenated or completely hydrogenated phospholipids and lecithins, lysolecithins and lysophosphatidylcholine, animal waxes, plant waxes, mineral waxes, synthetic waxes, wax esters, saturated and unsaturated fatty alcohols, fatty alcohol ethers/esters; solid saturated and unsaturated hydrocarbons (paraffins); solid silicones and silicone ethers/esters; polyol ethers/esters: glycerol ethers/esters, sorbitan, sorbitan stearate, glyceryl ricinoleate; polyglycerols and their ethers/esters, hydrophobic gelling agents: silicon dioxide, polyethylenes, or mixtures thereof.

In various embodiments, one or more of the solid fatty compounds is a wax, for example, a vegetable wax, an animal wax, a mineral, a synthetic wax and/or petroleum derived wax. Nonlimiting examples include Bayberry wax, candelilla wax, carnauba wax, castor wax, esparto wax, japan wax, ouricury wax, rice bran wax, soy wax, tallow tree wax, beeswax, Chinese wax, lanolin wax (wool wax), shellac wax, spermaceti wax, and mixtures thereof, paraffin wax, microcrystalline wax, ceresin wax, montan wax, ozocerite wax, polyethylene wax, peat wax.

In further embodiments, one or more of the solid fatty compounds may be a resin, for example, plant and/or animal and/or petroleum derived resin and/or a synthetic resin. Nonlimiting examples include asphaltite, Utah resin (petroleum bitumens), shellac, copals, dammars, mastic, sandarac, frankincense, elemi, turpentine, copaiba, gum resins, Aleppo pine resin, bisphenol A diglycidyl ether and silicone resins.

Solid fatty alcohols include those represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include cetyl alcohol, stearyl alcohol, behenyl alcohol and mixtures thereof, preferably cetyl alcohol, behenyl alcohol, cetearyl alcohol, and mixtures thereof.

Nonlimiting examples of solid fatty acids include decanoic acid, lauric acid, myristic acid, palmitic acid, stearic acid, arachidic acid or behenic acid. Solid fatty alcohols may be linear, unsaturated 1-alkanols with at least 12 carbon atoms. Examples of solid fatty alcohols are lauryl alcohol, myristyl alcohol, cetyl alcohol, stearyl alcohol, arachidyl alcohol, behenyl alcohol, or mixtures thereof.

In certain embodiments, the one or more solid fatty compounds are selected from waxes, fatty alcohols, solid fatty acids, or combinations thereof. Nonlimiting examples include candelilla wax, carnauba wax, castor wax, beeswax, lanolin Wax, ozokerite wax, microcrystalline wax, sunflower wax, tribehenin, cetyl alcohol, stearyl alcohol, cetearyl alcohol, lanolin alcohol, or mixtures thereof.

The total amount of the one or more solid fatty compounds, if present, will vary. Nonetheless, the second anhydrous composition may optionally include from about 0.01 to about 20 wt. % of one or more solid fatty compounds. In further embodiments, the second anhydrous composition comprises from about 0.01 to about 15 wt. %, about 0.01 to about 10 wt. %, about 0.01 to about 5 wt. %, about 0.01 to about 3 wt. %, of one or more solid fatty compounds. In other embodiments, the second anhydrous composition comprises from about 0.1 to about 20 wt. %, about 0.1 to about 15 wt. %, about 0.1 to about 10 wt. %, about 0.1 to about 5 wt. %, about 1 to about 20 wt. %, about 1 to about 15 wt. %, about 1 to about 10 wt. %, about 1 to about 5 wt. %, about 2 to about 20 wt. %, about 2 to about 15 wt. %, about 2 to about 10 wt. %, about 2 to about 5 wt. %, about 5 to about 20 wt. %, about 5 to about 15 wt. %, or about 5 to about 10 wt. % of one or more solid fatty compounds based on the total weight of the second anhydrous composition.

### Miscellaneous Ingredients

The second anhydrous composition optionally includes one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the second anhydrous composition, if present, will vary. Nonetheless, the second anhydrous composition may include from about 0.1 to about 15 wt. % of the one or more miscellaneous ingredients, based on the total weight of the second anhydrous composition. In further embodiments, the first anhydrous composition includes from about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of one or more miscellaneous ingredients, based on the total weight of the second anhydrous composition.

### EMBODIMENTS

In a preferred embodiment, the method for removing makeup from skin comprises, consists essentially of, or consists of
(i) applying a first anhydrous composition to skin upon which makeup is present, the first anhydrous composition comprising, consisting essentially of, or consisting of:
   (i)(a) about 1 to about 40 wt. %, preferably about 10 to about 35 wt. %, more preferably about 20 to about 35 wt. % a hydrophobic polymer that is a reaction product of a natural or food-derived oil and a methacrylate or acrylate polymer, wherein preferably
      the hydrophobic polymer is a reaction product of: (i) a natural or food-derived oil selected from linseed oil, sunflower oil, tung oil, fish oil, cottonseed oil, soybean oil, or combinations thereof, preferably linseed oil; and(ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
      the hydrophobic polymer is the reaction product of linseed oil and poly(isobutyl methacrylate);
   (i)(b) about 30 to about 80 wt. %, preferably from about 40 to about 75 wt. %, more preferably from about 45 to about 70 wt. % of one or more solvents capable of solubilizing the hydrophobic polymer of (i)(a), wherein preferably,
      the one or more solvents capable of solubilizing the reaction product of (i)(a) have a dispersion component (D), a polar component (β), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5}
P₁ is 4.8 MPa^{0.5}, and
H₁ is 13.0 MPa^{0.5}, wherein more preferably,
the one or more solvents capable of solubilizing the reaction product of (i)(a) have a dispersion component (D), a polar component (β), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5}
P₁ is 5.0 MPa^{0.5}, and
H₁ is 11.7 MPa^{0.5}, wherein even more preferably,
   at least one of the one or more solvents capable of dissolving the hydrophobic polymer of (i)(a) are selected from dioctylcyclohexane, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglyceride, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C₁₂-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C₁₂-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof; and
(i)(c) optionally, about 1 to about 30 wt. %, preferably about 5 to about 25 wt. %, more preferably about 10 to about 20 wt. % of one or more alkanes, wherein preferably the one or more alkanes are selected from selected from C₆-C₁₆ alkanes, wherein even more preferably,
   the one or more alkanes are selected from isododecane, isodecane, isohexadecane, n-heptane, n-octane, n-nonane, n-decane, n-undecane, n-dodecane, n-tetradecane, n-tridecane, n-tetradecane, n-pentadecane, or mixtures thereof;
(i)(d) optionally, one or more oil thickening agents, wherein if present, the one or more oil thickening agents are preferably selected from polymers, gums, organoclays, polyethylenes, silica, or combinations thereof, wherein, if present, may be in an amount of from about 0.01 to about 10 wt. %, preferably about 0.1 to about 5 wt. %, more preferably about 0.5 to about 4 wt. %,
(i)(e) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein all weight percentages of (i) are based on a total weight of the first anhydrous composition;
(ii) applying a second anhydrous composition to skin upon which the makeup and the first anhydrous composition are present, the second anhydrous composition comprising, consisting essentially of, or consisting of:
   (ii)(a) about 50 to about 100 wt. %, preferably from about 60 to about 99 wt. %, more preferably from about 75 to about 95 wt. % of one or more nonpolar oils in which the hydrophobic polymer of (i)(a) is not soluble, wherein preferably,
      the one or more nonpolar oils have a polarity index (interfacial tension) of at greater than 37 mN/m, wherein more preferably,
      the one or more nonpolar oils are selected from squalane, isoparaffin (C12-C14), cycloparaffin, polydecene, polydimethylsiloxane, squalene, hydrogenated polyisobutene, isohexadecane, mineral oil, isoeicosane, ethoxydiglycol oleate, decyl olivate, or mixtures thereof;
   (ii)(b) optionally, one or more oil thickening agents, wherein if present, the one or more oil thickening agents are preferably selected from polymers, gums, organoclays, polyethylenes, silica, or combinations thereof, wherein, if present, may be in an amount of from about 0.01 to about 10 wt. %, preferably about 0.1 to about 5 wt. %, more preferably about 0.5 to about 4 wt. %,
   (ii)(c) optionally, one or more solid fatty compounds, wherein if present, may be in an amount of from about 0.01 to about 20 wt. %, preferably about 0.1 to about 15 wt. %, more preferably about 1 to about 10 wt. %, wherein preferably, at least one of the one or more solid fatty compounds is a wax, for example, one or more waxes selected from candelilla wax, carnauba wax, castor wax, beeswax, lanolin Wax, ozokerite wax, microcrystalline wax, sunflower wax, tribehenin, or mixtures thereof,
   (ii)(d) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %.
      wherein all weight percentages of (ii) are based on the total weight of the second anhydrous composition.

The first anhydrous composition, the second anhydrous composition, or both the first anhydrous composition and the second anhydrous may be transparent or translucent.

In another preferred embodiment, the method for removing makeup from skin comprises, consists essentially of, or consists of
(i) applying a first anhydrous composition to skin upon which makeup is present, the first anhydrous composition comprising, consisting essentially of, or consisting of:
   (i)(a) about 1 to about 40 wt. %, preferably about 10 to about 35 wt. %, more preferably about 20 to about 35 wt. % a hydrophobic polymer that is a reaction product of linseed oil and poly(isobutyl methacrylate), wherein preferably,
      the hydrophobic polymer is the reaction product of about 50 to about 85 parts by weight of the linseed oil and about 15 to about 50 parts by weight of the poly(isobutyl methacrylate), wherein more preferably,
      the hydrophobic polymer is the reaction product of about 72 to about 77 parts by weight of the linseed oil and about 23 to about 28 parts by weight of the poly(isobutyl methacrylate);
   (i)(b) about 30 to about 80 wt. %, preferably from about 40 to about 75 wt. %, more preferably from about 45 to about 70 wt. % of one or more solvents capable of solubilizing the hydrophobic polymer of (i)(a), wherein preferably,
      the one or more solvents capable of solubilizing the reaction product of (i)(a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof, wherein more preferably,
      the one or more solvents capable of solubilizing the hydrophobic polymer of (i)(a) are selected from polar oils and medium polar oils having a Polarity Index (interfacial tension) of 37 mN/m or below, wherein even more preferably,
      the one or more solvents capable of solubilizing the hydrophobic polymer of (i)(a) are selected from polar oils having a Polarity Index (interfacial tension) of 23 to 37 mN/m;
   (i)(c) optionally, about 1 to about 30 wt. %, preferably about 5 to about 25 wt. %, more preferably about 10 to about 20 wt. % of one or more alkanes, wherein preferably the one or more alkanes are selected from selected from C₆-C₁₆ alkanes, wherein even more preferably,
      the one or more alkanes are selected from isododecane, isodecane, isohexadecane, n-heptane, n-octane, n-nonane, n-decane, n-undecane, n-dodecane, n-tetradecane, n-tridecane, n-tetradecane, n-pentadecane, or mixtures thereof, and even more preferably,
      at least one of the one or more alkanes is isododecane;
   (i)(d) optionally, one or more oil thickening agents, wherein if present, the one or more oil thickening agents are preferably selected from polymers, gums, organoclays, polyethylenes, silica, or combinations thereof, wherein, if present, may be in an amount of from about 0.01 to about 10 wt. %, preferably about 0.1 to about 5 wt. %, more preferably about 0.5 to about 4 wt. %,
   (i)(e) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
      wherein all weight percentages of (i) are based on a total weight of the first anhydrous composition;
(ii) applying a second anhydrous composition to skin upon which the makeup and the first anhydrous composition are present, the second anhydrous composition comprising, consisting essentially of, or consisting of:
   (ii)(a) about 50 to about 100 wt. %, preferably from about 60 to about 99 wt. %, more preferably from about 75 to about 95 wt. % of one or more nonpolar oils in which the hydrophobic polymer of (i)(a) is not soluble, wherein preferably,
      the one or more nonpolar oils have a polarity index (interfacial tension) of at greater than 37 mN/m, wherein more preferably,
      the one or more nonpolar oils are selected from squalane, isoparaffin (C12-C14), cycloparaffin, polydecene, polydimethylsiloxane, squalene, hydrogenated polyisobutene, isohexadecane, mineral oil, isoeicosane, ethoxydiglycol oleate, decyl olivate, or mixtures thereof, wherein even more preferably,
      at least one of the one or more nonpolar oils is squalane;
   (ii)(b) optionally, one or more oil thickening agents, wherein if present, the one or more oil thickening agents are preferably selected from polymers, gums, organoclays, polyethylenes, silica, or combinations thereof, wherein, if present, may be in an amount of from about 0.01 to about 10 wt. %, preferably about 0.1 to about 5 wt. %, more preferably about 0.5 to about 4 wt. %,
   (ii)(c) optionally, one or more solid fatty compounds, wherein if present, may be in an amount of from about 0.01 to about 20 wt. %, preferably about 0.1 to about 15 wt. %, more preferably about 1 to about 10 wt. %, wherein preferably, at least one of the one or more solid fatty compounds is a wax, for example, one or more waxes selected from candelilla wax, carnauba wax, castor wax, beeswax, lanolin Wax, ozokerite wax, microcrystalline wax, sunflower wax, tribehenin, or mixtures thereof;
   (ii)(d) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %.
   wherein all weight percentages of (ii) are based on the total weight of the second anhydrous composition.

The first anhydrous composition, the second anhydrous composition, or both the first anhydrous composition and the second anhydrous may be transparent or translucent.

In another preferred embodiment, the method for removing makeup from skin comprises, consists essentially of, or consists of
(i) applying a first anhydrous composition to skin upon which makeup is present, the first anhydrous composition comprising, consisting essentially of, or consisting of:
   (i)(a) about 1 to about 40 wt. %, preferably about 10 to about 35 wt. %, more preferably about 20 to about 35 wt. % of a reaction product of about 72 to about 77 parts by weight of the linseed oil and about 23 to about 28 parts by weight of the poly(isobutyl methacrylate);
   (i)(b) about 30 to about 80 wt. %, preferably from about 40 to about 75 wt. %, more preferably from about 45 to about 70 wt. % of one or more solvents capable of solubilizing the hydrophobic polymer of (i)(a), wherein preferably, at least one of the one or more solvents is polycitronellol acetate, caprylic capric triglycerine, isopropyl myristate, or combinations thereof;
   (i)(c) optionally, about 1 to about 30 wt. %, preferably about 5 to about 25 wt. %, more preferably about 10 to about 20 wt. % of one or more alkanes selected from isododecane, isodecane, isohexadecane, n-heptane, n-octane, n-nonane, n-decane, n-undecane, n-dodecane, n-tetradecane, n-tridecane, n-tetradecane, n-pentadecane, or mixtures thereof, and even more preferably, at least one of the one or more alkanes is isododecane;
   (i)(d) optionally, one or more oil thickening agents, wherein if present, the one or more oil thickening agents are preferably selected from polymers, gums, organoclays, polyethylenes, silica, or combinations thereof, wherein, if present, may be in an amount of from about 0.01 to about 10 wt. %, preferably about 0.1 to about 5 wt. %, more preferably about 0.5 to about 4 wt. %,
   (i)(e) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
      wherein all weight percentages of (i) are based on a total weight of the first anhydrous composition;
(ii) applying a second anhydrous composition to skin upon which the makeup and the first anhydrous composition are present, the second anhydrous composition comprising, consisting essentially of, or consisting of:
   (ii)(a) about 50 to about 100 wt. %, preferably from about 60 to about 99 wt. %, more preferably from about 75 to about 95 wt. % of one or more nonpolar oils in which the hydrophobic polymer of (i)(a) is not soluble, wherein preferably, the one or more nonpolar oils are selected from squalane, isoparaffin (C₁₂-C₁₄), cycloparaffin, polydecene, polydimethylsiloxane, squalene, hydrogenated polyisobutene, isohexadecane, mineral oil, isoeicosane, ethoxydiglycol oleate, decyl olivate, or mixtures thereof, wherein even more preferably, at least one of the one or more nonpolar oils is squalane;
   (ii)(b) optionally, one or more oil thickening agents, wherein if present, the one or more oil thickening agents are preferably selected from polymers, gums, organoclays, polyethylenes, silica, or combinations thereof, wherein, if present, may be in an amount of from about 0.01 to about 10 wt. %, preferably about 0.1 to about 5 wt. %, more preferably about 0.5 to about 4 wt. %,
   (ii)(c) optionally, one or more solid fatty compounds, wherein if present, may be in an amount of from about 0.01 to about 20 wt. %, preferably about 0.1 to about 15 wt. %, more preferably about 1 to about 10 wt. %, wherein preferably, at least one of the one or more solid fatty compounds is a wax, for example, one or more waxes selected from candelilla wax, carnauba wax, castor wax, beeswax, lanolin Wax, ozokerite wax, microcrystalline wax, sunflower wax, tribehenin, or mixtures thereof,
   (ii)(d) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %.
   wherein all weight percentages of (ii) are based on the total weight of the second anhydrous composition.

The first anhydrous composition, the second anhydrous composition, or both the first anhydrous composition and the second anhydrous may be transparent or translucent.

### EXAMPLES

Implementation of the present disclosure is provided by way of the following examples. The examples serve to illustrate the technology without being limiting in nature.

### Example 1

### Solubility Testing

Testing was carried out to ascertain the solubility of MyceIX® in various solvent mixtures of polycitronellol acetate and squalane. To determine how the solvent mixtures influence solubility of MycelX®, the MyceIX® was initially dissolved in polycitronellol acetate to form a transparent solution. Squalane was subsequently added to the solution in various amounts to assess how the addition of a nonpolar oil influences the solubility of the MyceIX®. When polycitronellol acetate and squalane were combined in a weight ratio of 80/20 (MycelX®:squalane), the MyceIX® was soluble in all amounts tested, i.e., 30 wt. %, 40 wt. %, 50 wt. %, and 60 wt. %. When polycitronellol acetate and squalane were combined in a weight ratio of 65:35 (polycitronellol acetate:squalane), the combination solubilized 10 wt. % of the MyceIX® but only partially solubilized 20 wt. % of the MyceIX®. At 20 wt. % MycelX®, the mixture became hazy (lost transparency) indicating a degree of precipitation. At 30 and 40 wt. % MyceIX®, the MyceIX® precipitated resulting in hazy mixture with a yellow color, which contained particles of the MyceIX®. The results are summarized in the table below.

| | | Solubility of MycelX ® | | | |
|---|---|---|---|---|---|
| | | Ratio of MycelX ®/Squalane (wt. %) | | | |
| | | 80/20 | 65/35 | 50/50 | 15/85 |
| MYCELEX ® | 60 | Soluble | | | |
| (wt. %) | 50 | Soluble | | Not Soluble | |
| | 40 | Soluble | Not Soluble | Not Soluble | Not Soluble |
| | 30 | Soluble | Not Soluble | Not Soluble | Not Soluble |
| | 20 | | Partially | Not Soluble | Not Soluble |
| | | | Soluble | | |
| | 10 | | Soluble | | Not Soluble |

### Example 2

### Peeling Studies

Various compositions were prepared and studied to determine their peel properties in comparison to a commercial-peeling facial cleanser, used as a benchmark. The compositions are shown in the following table.

| | First Anhydrous Composition | | | |
|---|---|---|---|---|
| | Inventive | Comparative | | Benchmark |
| | A | B | C | E |
| MycelX ®¹ | 28.6 | | | * |
| Polymethyl Methacrylate | | 28.6 | | |
| Linseed Oil | | | 28.6 | |
| Polycitronellol Acetate | 57.1 | 57.1 | 57.1 | |
| Isododecane | 14.3 | 14.3 | 14.3 | |

¹Reaction product of linseed oil and isobutyl methacrylate polymer
* The commercial benchmark product reports the following ingredients list: Aqua/Water, Glycerin, Dicocodimonium Chloride, Carbomer, Steartrimonium Bromide, Isopropyl Alcohol, Dipotassium Glycyrrhizate, Allantoin, Citric Acid, Malic Acid, Lactic Acid, Sodium Hyaluronate, Aloe Barbadensis Leaf Extract, Punica Granatum Pericarp Extract, Soluble Collagen, Placental Protein, Fucus Vesiculosus Extract, Clematis Vitalba Leaf Extract, Spiraea Ulmaria Flower Extract, Equisetum Arvense (Horsetail) Extract, Hedera Helix (Ivy) Extract, Butylene Glycol, Phenoxyethanol, Fragrance; Parfum; Aroma, CI 16255; ACID RED 18.

About 2 μL of matte ink long-wear makeup (red) was uniformly applied onto white supplare substrates (2×2 inch) and dried in a 37° C. chamber for about 1 hour, or until dry. After drying, about 2 μL of inventive composition A, comparative composition B, comparative composition C (control), and the commercial benchmark were applied with pipette to separate dried makeup samples and rubbed with a finger over the makeup in a circular motion ten times. After waiting about 10 seconds, about 2 μL of squalane (second anhydrous composition) was subsequently applied with pipette to the makeup samples and rubbed with a fiber over the makeup in a circular motion ten times. Upon application of the squalane to the makeup sample treated with inventive composition A, the makeup coagulated and peeled from the supplare substrate. The makeup samples treated with comparative compositions B, comparative composition C, and the commercial benchmark E did not coagulate and peel from the supplare substrate upon application of the squalane. Additionally, water was added on top of benchmark E, but there is very minimal peeling.

### Example 3

### Makeup Removing Studies

Various compositions were prepared and studied to determine their ability to remove makeup in comparison to a commercial benchmark makeup removing product. The compositions are shown in the following table. Inventive composition A, comparative compositions B and C, and the commercial benchmark product E are identical to those tested in Example 2.

| | First Anhydrous Composition | | | | |
|---|---|---|---|---|---|
| | Inventive | Comparative | | | Benchmark |
| | A | B | C | D | E |
| MycelX ®¹ | 28.6 | | | | * |
| Polymethyl | | 28.6 | | | |
| Methacrylate | | | | | |
| Linseed Oil | | | 28.6 | | |
| Polybutene | | | | 58.6 | |
| Polycitronellol | 57.1 | 57.1 | 57.1 | 57.1 | |
| Acetate | | | | | |
| Isododecane | 14.3 | 14.3 | 14.3 | 14.3 | |

¹Reaction product of linseed oil and isobutyl methacrylate polymer
* The commercial benchmark product reports the following ingredients list: Aqua/Water, Cyclopentasiloxane, Isohexadecane, Sodium Chloride, Poloxamer 184, Hexylene Glycol, Dipotassium Phosphate, Benzyl Alcohol, Potassium Phosphate, Quaternium-15, Benzalkonium Chloride, Parfum/Fragrance, Citronellol, Geraniol.

About 20 μL of matte ink long-wear makeup (red) was uniformly applied onto a black supplare substrates (1.25×1.25 inch) and dried in a 37° C. chamber for about 1 hour or until dry. After drying, about 20 μL of inventive composition A, comparative composition B, comparative composition C (control), comparative composition D, and the commercial benchmark product E were applied by pipette to separate dried makeup samples and rubbed over the makeup in a circular motion ten times. After waiting about 10 seconds, about 20 μL of squalane (second anhydrous composition) was subsequently applied by pipette to the makeup samples and rubbed with a finger over the makeup in a circular motion ten times. For the sample treated with the commercial benchmark product, instead of squalane, an additional about 20 μL of the commercial benchmark product was reapplied in the second step. The samples were then wiped with a cotton pad under controlled pressure using a pressure sensor control the pressure at around 500 gram force L*a*b* values were measured using a Datacolor 600™ Spectraflash spectrometer. The percent removal of makeup was calculated according to the following formula. Each sample was run in triplicate and the results averaged.

$$\% Removal = ( a {*}_{( after )} - a {*}_{( before} ) / ( a {*}_{( before )} - a {*}_{( substrate )} )$$

% Removal (L*a*b*)

| Inventive | Comparative | | | Benchmark |
|---|---|---|---|---|
| A | B | C | D | E |
| 84.4 | 76 | 61.7 | 66.0 | 75.1 |

The results show that inventive composition A removed the highest percentage of the long wear makeup, even more than the commercial benchmark standard.

### Example 4

### Makeup Removing Studies

Various compositions were prepared and studied to determine their ability to remove makeup in comparison to a commercial benchmark product for removing makeup. The compositions are shown in the following table. Inventive composition A and the commercial benchmark product E are identical to those studied in Examples 2 and 3.

| | First Anhydrous Composition | | | |
|---|---|---|---|---|
| | Inventive | Comparative | | Benchmark |
| | A | F | G | E |
| MycelX ®¹ | 28.6 | | | * |
| Polycitronellol Acetate | 57.1 | | | |
| Caprylic/capric triglycerides | | 57.1 | | |
| Isopropyl Myristate | | | 57.1 | |
| Isododecane | 14.3 | 14.3 | 14.3 | |

¹Reaction product of linseed oil and isobutyl methacrylate polymer
* The commercial benchmark product reports the following ingredients list: Aqua/Water, Cyclopentasiloxane, Isohexadecane, Sodium Chloride, Poloxamer 184, Hexylene Glycol, Dipotassium Phosphate, Benzyl Alcohol, Potassium Phosphate, Quaternium-15, Benzalkonium Chloride, Parfum/Fragrance, Citronellol, Geraniol.

About 20 μL of matte ink long-wear makeup (red) was uniformly applied onto black supplare substrates (1.25×1.25 inch) and dried in a 37° C. chamber for about 1 hour, or until dry. After drying, about 20 μL of inventive compositions A-D or the commercial benchmark product E were applied by pipette to separate dried makeup samples and rubbed over the makeup with a finger in a circular motion ten times. After waiting about 10 seconds, about 20 μL of squalane (second anhydrous composition) was subsequently applied by pipette to the makeup samples. For the sample treated with the commercial benchmark product, instead of squalane, an additional about 20 μL of the commercial benchmark product was reapplied by pipette in the second step. The samples were then wiped with a cotton pad under controlled pressure using a SWIFT Tool in the green zone. L*a*b* values were measure using a Datacolor 600™ Spectraflash spectrometer. The percent removal of makeup was calculated and according to the following formula. Each composition was tested in triplicate and the results averaged.

$$\% Removal = ( a {*}_{( after )} - a {*}_{( before} ) / ( a {*}_{( before )} - a {*}_{( substrate )} )$$

% Removal (L*a*b*)

| Inventive | | | Benchmark |
|---|---|---|---|
| A | F | G | E |
| 84.4 | 40.5 | 63.8 | 75.1 |

The results show that inventive composition A removed significantly more long wear makeup, even more than the commercial benchmark standard.

### Example 5

### Smudge Testing

Various compositions were prepared and studied to determine their performance using a smudge test in comparison to a commercial benchmark product for removing makeup. The compositions are shown in the following table. Inventive composition A, comparative composition C, and the commercial benchmark product are identical to those studied in Examples 2-4.

| | First Anhydrous Composition | | |
|---|---|---|---|
| | Inventive | Comparative | Benchmark |
| | A | C | E |
| MycelX ®1 | 28.6 | | * |
| Linseed Oil | | 28.6 | |
| Polycitronellol Acetate | 57.1 | 57.1 | |
| Isododecane | 14.3 | 14.3 | |

¹Reaction product of linseed oil and isobutyl methacrylate polymer
* The commercial benchmark product reports the following ingredients list: Aqua/Water, Cyclopentasiloxane, Isohexadecane, Sodium Chloride, Poloxamer 184, Hexylene Glycol, Dipotassium Phosphate, Benzyl Alcohol, Potassium Phosphate, Quaternium-15, Benzalkonium Chloride, Parfum/Fragrance, Citronellol, Geraniol.

Two different waterproof, long-wear commercially available mascaras products were used in the testing, Mascara 1 and Mascara 2. The mascara products were each applied fifteen times to eyelash samples. The eyelash samples were then placed on top of white supplar. About 4 μL of inventive composition A or the commercial benchmark product E were applied by pipette to eyelash samples treated with Mascara 1 and separately about 4 μL of inventive composition A and comparative compositions C were applied by pipette to eyelash samples treated with Mascara 2. The eyelash samples were rubbed by hand ten times from left to right followed by rubbing ten times from front to back. After rubbing, about 4 μL of squalane was applied by pipette to the eyelash samples treated with inventive composition A and comparative composition C. For the eyelash samples treated with the commercial benchmark product, instead of squalane, an additional about 4 μL of the commercial benchmark product was reapplied in the second step. Again, the eyelash samples were rubbed by hand ten times from left to right followed by rubbing ten times from front to back. A round cotton pad was placed underneath the attachment of a texture analyzer (Stable Micro System). A pressure of 150 g was applied over the eyelash samples and the eyelash samples were pulled over the cotton pad for a 5 second period. L*a*b* values were measured using a VeriVide DigiEye® Imaging System and L values compared. The higher the L value, the brighter/whiter the sample. Each composition was tested in triplicate and the results averaged. ←Is this correct?

% Removal Mascara 1

| Inventive | Benchmark |
|---|---|
| A | E |
| 77.2 ± 2.9 | 63.4 ± 6.7 |

% Removal Mascara 2

| Inventive | Comparative |
|---|---|
| A | C |
| 86.0 ± 1.5 | 79.4 ± 1.1 |

The results show that inventive composition A removed significantly more long wear makeup than the commercial benchmark and comparative composition C.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure.

The term “translucent” with respect to a translucent composition indicates that the composition has a transmittance of at least 50% at a wavelength of 600 nm, for example measured using a Lambda 40 UV-visible spectrometer.

Other than in the operating examples, or where otherwise indicated, all amount expressing quantities of ingredients and/or reaction conditions may be modified in all instances by the term “about,” meaning within +/−5% of the indicated number. Thus, for a range of “about 1 to about 10 wt. %,” The lower amount of “about 1 wt. %” may extend down to 0.95 wt. %, which is 5% less than 1 wt. %. The higher amount of “about 10 wt. %” may extend up to 10.5 wt. %, which is 5% higher than 10 wt. %, i.e., a range of “0.95 wt. % to 10.5 wt. %.”

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both oily solvent and a surfactant. If a particular composition includes both an oily solvent and a surfactant, a single compound will serve as only the oily solvent or only as the surfactant (the single compound does not simultaneously serve as both the oily solvent and the surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. For example, if a composition is essentially free from compound X, the composition includes less that 2 wt. % of compound X, or less than 1 wt. % of compound X, or less than 0.5 wt. % of compound X, or less than 0.1 wt. % of compound X, or is free from compound X.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for removing makeup from skin comprising:
(i) applying a first anhydrous composition to skin upon which makeup is present, the first anhydrous composition comprising:
(a) a hydrophobic polymer formed as a reaction product of a natural or food-derived oil and a methacrylate or acrylate polymer;
(b) one or more solvents capable of solubilizing the hydrophobic polymer of (i)(a); and
(c) optionally, one or more alkanes;
(ii) applying a second anhydrous composition to skin upon which the makeup and the first anhydrous composition are present, the second anhydrous composition comprising:
(d) one or more nonpolar oils in which the hydrophobic polymer of (i)(a) is not soluble.

2. The method of claim 1, wherein the first anhydrous composition is transparent.

3. The method of claim 1, the natural or food-derived oil is a drying oil or semi-drying oil selected from linseed oil, sunflower oil, tung oil, fish oil, cottonseed oil, soybean oil, or combinations thereof, and the methacrylate or acrylate polymer is derived from monomers selected from isobutyl methacrylate, methyl methacrylate, ethyl methacrylate, n-butyl methacrylate, or combinations thereof.

4. The method of claim 1, wherein the hydrophobic polymer is the reaction product of about 50 to about 85 parts by weight of the natural or food-derived oil and about 15 to about 50 parts by weight of the methacrylate or acrylate polymer.

5. The composition of claim 1, wherein the methacrylate or acrylate polymer is an isobutyl methacrylate polymer.

6. The composition of claim 1, wherein the hydrophobic polymer is a reaction product of linseed oil and poly(isobutyl methacrylate).

7. The composition of claim 6, wherein the hydrophobic polymer is the reaction product of about 72 to about 77 parts by weight of linseed oil and about 23 to about 28 parts by weight of isobutyl methacrylate polymer.

8. The method of claim 1, wherein the one or more solvents capable of solubilizing the hydrophobic polymer of (i)(a) have a dispersion component (D), a polar component (P), and a hydrogen bonding component (H), and a distance (Ra) less than or equal to 13.4 MPa^{0.5} as per Hansen Solubility Parameters, wherein the distance (Ra) is defined by formula (I):
$$Ra = \sqrt{4 {( D - {D}_{1} )}^{2} + {( P - {P}_{1} )}^{2} + {( H - {H}_{1} )}^{2}} \qquad ( I )$$
wherein
D₁ is 16.8 MPa^{0.5},
P₁ is 4.8 MPa^{0.5}, and
H₁ is 13.0 MPa^{0.5}.

9. The composition of claim 1, wherein at least one of the one or more solvents capable of solubilizing the hydrophobic polymer of (i)(a) is selected from polycitronellol acetate, caprylic/capric triglyceride, isododecane, isohexadecane, tetradecane, isopropyl myristate, isopropyl alcohol, octyldodecanol, phenoxyethanol, castor oil, or mixtures thereof.

10. The method of claim 1, wherein the first anhydrous composition comprises:
(a) about 10 to about 40 wt. % of the hydrophobic polymer;
(b) about 45 to about 75 wt. % of the one or more solvents capable of solubilizing the hydrophobic polymer of (i)(a); and
(c) about 1 to about 25 wt. % of the one or more alkanes;
wherein all weight percentages are based on a total weight of the first anhydrous composition.

11. The method of claim 1, wherein the one or more solvents capable of solubilizing the hydrophobic polymer of (i)(a) are selected from dioctylcyclohexane, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether isododecane, isodecyl 3,5,5 trimethyl hexanoate, oleyl erucate, Passiflora incarnata oil, Jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic scid 3,5,5 trimethyl ester, cetystearyl octanoate, dimethicone, isopropyl palmitate octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, cyclomethicone, groundnut oil, almond oil, sunflower oil, decyl oleate, avocado oil, olive oil, dibutyl adipate, castor oil, calendula oil, wheatgerm oil, sunflower oil, decyl oleate, castor oil, calendula oil, wheatgerm oil, propylene glycol monoisostearate, cocoglycerides, or mixtures thereof.

12. The method of claim 1, wherein the first anhydrous composition comprises the one or more alkanes and the one or more alkanes are selected from C₆-C₁₆ alkanes, preferably wherein the one or more alkanes are selected from isododecane, isodecane, isohexadecane, n-heptane, n-octane, n-nonane, n-decane, n-undecane, n-dodecane, n-tetradecane, n-tridecane, n-tetradecane, n-pentadecane, or mixtures thereof.

13. The method of claim 1, wherein the first anhydrous composition comprises:
(a) about 10 to about 40 wt. % of the hydrophobic polymer;
(b) about 45 to about 75 wt. % of polycitronellol acetate; and
(c) about 1 to about 25 wt. % of isododecane;
wherein all weight percentages are based on a total weight of the first anhydrous composition.

14. The method of claim 1, wherein the one or more nonpolar oils in which the hydrophobic polymer of (i)(a) is not soluble are selected from squalane, isoparaffin (C12-C14), cycloparaffin, polydecene, polydimethylsiloxane, squalene, hydrogenated polyisobutene, isohexadecane, mineral oil, isoeicosane, ethoxydiglycol oleate, decyl olivate, or mixtures thereof.

15. The method of claim 1, wherein the second anhydrous composition further comprises one or more oil thickening agents, for example, one or more oil thickening agents selected from polymers, gums, organoclays, polyethylenes, silica, or mixtures thereof, more preferably wherein the one or more oil thickening agents are selected from acrylate copolymer, hectorite gel, guar gum, cellulose gum, Caesalpinia Spinosa gum, gum Arabic, starch, silica, silica dimethyl silylate, behenate, glyceryl dibehenate, behenyl behenate, or mixtures thereof.

16. The method of claim 1, wherein the second anhydrous composition further comprises one or more solid fatty compounds, wherein preferably the one or more solid fatty compounds selected from waxes and solid fatty alcohols, for example, candelilla wax, carnauba wax, castor wax, beeswax, lanolin Wax, ozokerite wax, microcrystalline wax, sunflower wax, tribehenin, cetyl alcohol, stearyl alcohol, cetearyl alcohol, lanolin alcohol, or mixtures thereof.

17. The method of claim 1, wherein the second anhydrous composition comprises:
(a) about 70 to about 99.9 wt. % of the one or more nonpolar oils in which the hydrophobic polymer of (i)(a) is not soluble;
(b) optionally, about 1 to about 15 wt. % of one or more oil thickening agents; and
(c) optionally, about 1 to about 15 wt. % of one or more waxes;
wherein all weight percentages are based on a total weight of the second anhydrous composition.

18. A method for removing makeup from skin comprising:
(i) applying a first anhydrous composition to skin upon which makeup is present, the first anhydrous composition comprising:
(a) about 10 to about 40 wt. % of a hydrophobic polymer that is a reaction product of linseed oil and poly(isobutyl methacrylate);
(b) about 45 to about 75 wt. % of polycitronellol acetate; and
(c) about 1 to about 25 wt. % of isododecane;
wherein all weight percentages of (i) are based on a total weight of the first anhydrous composition,
(ii) applying a second anhydrous composition to skin upon which the makeup and the first anhydrous composition are present, the second anhydrous composition comprising:
(a) about 70 to 95 wt. % of one or more nonpolar oils in which the hydrophobic polymer of (i)(a) is not soluble;
(b) optionally, about 1 to about 15 wt. % of one or more oil thickening agents; and
(c) optionally, about 1 to about 15 wt. % of one or more waxes;
wherein all weight percentages of (ii) are based on a total weight of the second anhydrous composition.

19. The method of claim 18, wherein the first anhydrous composition is transparent.

20. A makeup removing kit comprising:
(i) first anhydrous comprising:
(a) a hydrophobic polymer formed as a reaction product of a natural or food-derived oil and a methacrylate or acrylate polymer;
(b) one or more solvents capable of solubilizing the hydrophobic polymer of (i)(a); and
(c) optionally, one or more alkanes;
(ii) a second anhydrous composition comprising:
(d) one or more nonpolar oils in which the hydrophobic polymer of (i)(a) is not soluble;
wherein the first anhydrous composition and the second anhydrous composition are separately contained.
